(19) United States
(12) Patent Application Publication
(10) Pub. No.: US 2006/0247324 A1
(43) Pub. Date: Nov. 2, 2006

(54) Paper products softening compositions

(75) Inventors: Duy T Nguyen, Austin, TX (US); Samir S. Ashrawi, The Woodlands, TX (US)

Correspondence Address: Huntsman Corporation;Legal Department, 10003 Woodloch Forest Drive, The Woodlands, TX, 77380, US

(73) Assignee: Huntsman Petrochemical Corporation, The Woodlands, TX (US)

(21) Appl. No.: 10/545,858

(22) PCT Filed: Feb. 13, 2004
(86) PCT No.: PCT/US04/04337

§ 371 Date: Jun. 15, 2006

Related U.S. Application Data
(63) Continuation of application No. 10/369,263, filed on Feb. 18, 2003, now abandoned.

(51) Int. Cl. B01F 17/10; C07C 233/00; C07C 309/04

(52) U.S. Cl. 516/203; 562/30; 564/123

(57) ABSTRACT

Provided herein are amphoteric surfactants derived from ethyleneamines, which surfactants are useful in treating paper, fibers, textiles, hair, and human skin, to impart softness-to-the-touch properties thereto.

### TECHNICAL FIELD

This invention relates to compositions useful for treating various surfaces including fibers, textiles, paper, hair, and human skin. More particularly, it relates to compositions and methods for treating metal, paper, and textiles which compositions comprise an amphoteric surfactant derived from ethyleneamines, long-chain fatty acids, and acrylic acid. According to one preferred form of the invention the ethyleneamine used as a raw material from which the surfactant is derived is tetraethylenepentamine.

### BACKGROUND

U.S. Pat. No. 5,322,630 provides a method of acidizing a subterranean formation with an acqueous acid solution wherein the acid solution contains corrosion inhibiting amounts of an amine derivative prepared by reacting an unsaturated carboxylic acid with (a) fatty amine or polyamine, or (b) a fatty amido amine or polyamine, or (c) a fatty imidazoline amine or polyamine. The derivative is characterized by the absence of primary amino groups, and preferably contains only tertiary amino groups. Disclosed therein are amphoteric derivatives of a broad range of fatty polyamines, fatty amidoamines, fatty imidazolines and polyamines which are disclosed as being useful as oilfield corrosion inhibitors.

U.S. Pat. Nos. 6,004,914; 6,200,938; and 6,369,007 teach arnphoteric derivatives of aliphatic polyamines, such as diethylenetriamine or triethylenetetramine reacted with long chain fatty acids, esters or triglycerides from various natural or synthetic sources are effective in the softening/texture modification of substrates such as paper, textiles, human skin surfaces and hair tresses, as well as in applications for metal working and lubrication. The polyamines are first reacted with fatty acids, esters or triglycerides derived from various animal, vegetable or synthetic sources ranging in molecular distribution from butyric through erucic acids (e.g. milkfat, soy bean oil, rapeseed oil) to form polyamines or imidazolines; they are then further reacted with unsaturated or halogenated carboxylic acids, carboxylated epoxy compounds or acid anhydrides (e.g. acrylic acid, itaconic acid, chloroacetic acid, maleic anhydrides octadecenyl anhydride) to form the various amphoteric structures.

### SUMMARY OF THE INVENTION

The present invention relates to amphoteric surfactants that are useful in various applications including paper softener, fabric softener, metal working and lubrication. An amphoteric surfactant of the present invention may be made by reacting tetraethylene pentamine (“TEPA”) with 2.5 to 3.0 moles of a fatty acid to form an intermediate amide compound which is then converted to an amphoteric compound by reacting it with 1 to 2 moles of an unsaturated acid species selected from the group consisting of: maleic acid, maleic anhydride, vinyl sulfonic acid, 2-methyl vinyl sulfonic acid, allylsulfonic acid, and acrylic acid. Thus, the present invention concerns compositions of matter useful for treating paper, textiles, and human skin comprising an amphoteric surfactant represented by the formula: 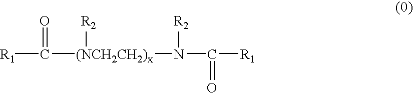
in which x is any integer selected from the group consisting of: 4, 5, and 6; R₁ in each occurrence is independently any alkyl group having between 5 and 25 carbon atoms, whether straight-chain, branched, cyclic, saturated or unsaturated; R₂ in each occurrence is independently selected from the group consisting of: 1) hydrogen; 2) any saturated or unsaturated aliphatic mono- or di-carboxylic acid moiety having one or more carboxyl functional groups and having one or more straight-chain or branched, saturated or un-saturated aliphatic chains containing from 2 to 20 carbon atoms; 3) any saturated or unsaturated aliphatic mono sulfonic acid moiety having one or more —SO₃H functional groups and having one or more straight-chain or branched, saturated or un-saturated aliphatic chains containing from 2 to 20 carbon atoms; and 4) a radical of the formula: 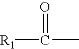
in which R₁ has the same meaning as that ascribed to it above.

According to another embodiment, a composition according to the invention comprises a mixture of at least two components each of which comprise different amphoteric surfactants which are represented by the formula: 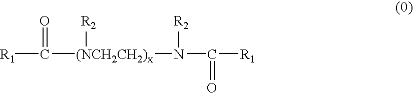
in which R₁ in each occurrence is independently any alkyl group having between 5 and 25 carbon atoms, whether straight-chain, branched, cyclic, saturated or unsaturated; R₂ in each occurrence is independently selected from the group consisting of: 1) hydrogen; 2) any saturated or unsaturated aliphatic mono- or di-carboxylic acid moiety having one or more carboxyl functional groups and having one or more straight-chain or branched, saturated or un-saturated aliphatic chains containing from 2 to 20 carbon atoms; 3) any saturated or unsaturated aliphatic mono sulfonic acid moiety having one or more —SO₃H functional groups and having one or more straight-chain or branched, saturated or un-saturated aliphatic chains containing from 2 to 20 carbon atoms; and 4) a radical of the formula: 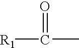
in which R₁ has the same meaning as that ascribed to it above. According to yet a further embodiment, the above-described mixture comprises:

a) a first amphoteric surfactant, having a value for x of 4;

b) a second amphoteric surfactant, having a value for x of 5;

c) a third amphoteric surfactant, having a value for x of 6,

with the first amphoteric surfactant being present in any amount between 8.0% and 20.0%; the second amphoteric surfactant being present in any amount between 25.0% and 45.0%; and the third amphoteric surfactant being present in any amount between 35.0% and 60.0%, with all percentages being calculated on a weight basis with respect to all of the amphoteric surfactants present which are defined by the above formula.

### BRIEF DESCRIPTION OF THE DRAWING

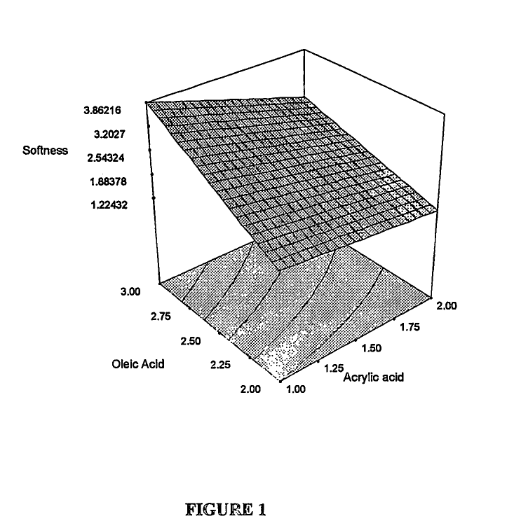

In the annexed drawing, FIG. 1 shows softness test results using of oleic acid and acrylic acid according to the present invention.

### DETAILED DESCRIPTION

An amphoteric surfactant of the present invention is exemplified by the use of TEPA as a raw material, and other amphoteric surfactants according to the invention are readily prepared using the same general procedure but with ethyleneamines such as pentaethylenehexamine, hexaethyleneheptamine, heptaethyleneoctamine, etc. An amphoteric surfactant according to the invention may be prepared by first reacting TEPA as a starting material with 2.5 to 3 moles fatty acids, to form an intermediate substituted TEPA polyamide. According to one preferred form of the invention, 3 moles of fatty acid are reacted with 1 mole of TEPA to yield the triamide. According to a preferred form of the invention, the polyamide is subsequently reacted with 1 to 2 moles of an unsaturated acid species such as acrylic acid or vinylsulfonic acid to form an amphoteric surfactant. According to one preferred form of the invention, 2 moles of acrylic acid are reacted with one mole of polyamide, which is preferably a triamide. The resulting amphoteric compounds are useful as softeners for tissue paper, fabrics, hair and skin. The resulting amphoteric compounds are also useful as lubricants in metalworking.

The general reaction scheme for producing an amphoteric surfactant useful in accordance with the present invention is set forth below: 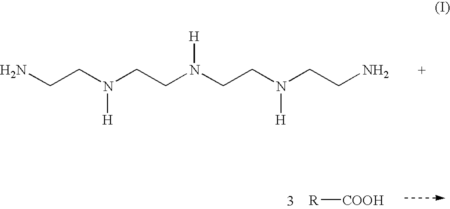
In reaction (1), one mole of tetraethylenepentamine is caused to be reacted with three moles of the mono-carboxylic acid in which R may be any C₁ through C₂₅ alkyl group, whether straight-chain, branched, cyclic, saturated or unsaturated. In the case of unsaturated carboxylic acids used as reactant with TEPA, the present invention contemplates the use of both cis- and trans- isomers. According to one preferred form of the invention, the reactant carboxylic acid is oleic acid, although any other carboxylic acid having between about 7 and 25 carbon atoms may be used, or mixtures thereof. The product of the reaction between three moles of the carboxylic acid and TEPA is the triamide shown in formula (II): 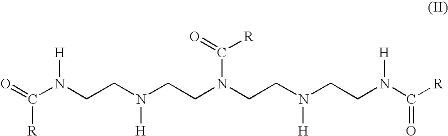
in which the R portion is supplied by the oleic acid.

This structure represents the predominant product of such reaction according to the invention. In practice, a mixture of positional isomers is formed with the carboxylic acid residue being substituted upon the various possible positions of substitution having an active hydrogen atom at which the acid function of the carboxylic acid is capable of reacting, as is known to those skilled in the art. When fewer than three moles of acid are reacted per mole of TEPA, the resulting product is a mixture of isomers substituted at the first and second; first and third; first and fourth; first and fifth; second and third; and second and fourth positions. The present invention embraces all such positional isomers and mixtures thereof.

Subsequent reaction of the polyamide shown in formula (II) with an unsaturated acid, such as, but not limited to, acrylic acid according to the formula (III): 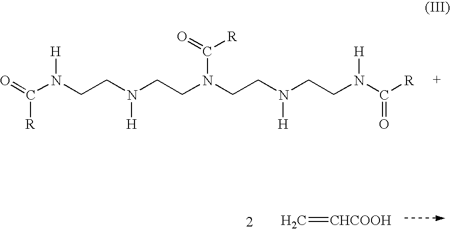
yields an amphoteric surfactant according to the invention, as described generally by formula (0) previously shown, and shown structurally in formula (IV): 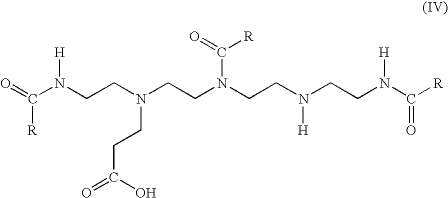
for the case where one mole of acrylic acid is reacted. When an unsaturated sulfonate such as vinylsulfonic acid or allylsulfonic acid is employed, the carboxylic acid group in the above structure is replaced by the group —SO₃H thus providing an amphoteric surfactant with a sulfonate anionic portion. The structure above represents the predominant product of such reaction according to the invention. In practice, a mixture of positional isomers is formed with the acrylic residue being substituted upon the various possible positions of substitution having an active hydrogen atom at which the unsaturated function of the acrylic acid is capable of reacting, as is known to those skilled in the art. When more than one mole of acrylic or other unsaturated carboxylic or sulfonic acid is reacted, more than one of the possible positions is substituted. The present invention embraces all such positional isomers. Monomers other than acrylic acid may of course be employed in the role just described for acrylic acid, including unsaturated acid species selected from the group consisting of: maleic acid, maleic anhydride, vinyl sulfonic acid, 2-methyl vinyl sulfonic acid, and allylsulfonic acid.

According to one preferred form of the invention, oleic acid is reacted with TEPA at 144° C. for about 6-10 hours and is subsequently reacted with acrylic acid in the presence of propylene glycol or polyethylene glycol at about 105° C. for about 8 hours, or until the reaction is complete. The structures of the reaction product are easily confirmed using NMR and IR spectroscopy.

The following examples are illustrative of the present invention and should not be construed as being delimitive thereof in any way. In general, any polyalkylene polyamine can be reacted with a fatty acid to yield an amide which is subsequently reacted with acrylic acid to yield an amphoteric surfactants useful in treating hair, skin, paper, textiles and fibers according to the invention.

### EXAMPLE 1

### Preparation of TEPA +3 Moles Oleic Acid (TEPA Triamide)

505.8 grams (1.8 moles) of oleic acid is charged to a 1 L round bottom flask equipped with a mechanical stirrer and nitrogen purge. 113.6 grams (0.60 moles) tetraethylene pentamine (“TEPA”) is slowly added with stirring under nitrogen at such a rate that the temperature is not permitted to exceed 120° C. Following the addition the temperature of the contents of the flask are maintained at 120° C. for 30 minutes, after which time the heat is increased to cause the reactor contents to reach 144° C., at which temperature the reactor contents are maintained for 6 hours further. Condensate is collected in a Dean-Stark trap (theoretical=32.4 ml). The reaction is considered to be complete when the acid number is below 10 meq/gram (acid numbers referred to in this specification are measured by titrating an aqueous sample using aqueous base which is about 0.1 N to a phenolphthalein end point and calculating the acid number using the relation:
meq/gram=((B)×(N)×56.1)/(weight of sample in grams)
in which B=the total number of milliliters of base used; and

N=the Normality of the base used.

The resulting product is a waxy solid at room temperature. Total yield=93.0% of theoretical, as determined by NMR and IR spectra. The resulting product is a waxy solid at room temperature. Total yield=93.0% of theoretical, as determined by NMR and IR spectra.

### EXAMPLE 2

### Preparation of TEPA Triamide Amphoteric Surfactant

To a 3-neck 1 L round bottom flask equipped with a mechanical stirrer, nitrogen purge, and addition funnel is charged 130.6 grams of propylene glycol and 98.3 grams (0.1 moles) of the oleic acid triamide of TEPA prepared from example 1 above. The contents of the flask are heated with stirring to 90° C. until the contents became homogeneous. 7.2 grams (0.1 mole) of acrylic acid are added slowly, and the contents of the flask are maintained at 105° C. for 3 hours. Alternatively, the reaction may be terminated when at least 90% of the acrylic acid has reacted, as determined by quantitative IR spectroscopy.

### EXAMPLE 3

### Preparation of Ethyleneamine E-100+3 Moles TOFA (E-100 Triamide)

Ethyleneamine E-100 (Huntsman Corp.) is a mixture of tetraethylenepentamine (10-15% TEPA), pentaethylenehexamine (33-38% PEHA) and hexaethyleneheptamine (45-54% HEHA). 516.4 grams of tall oil fatty acid (“TOFA”) is charged to a IL round bottom flask. under nitrogen purge. 162.6 grams of Ethylenamine E-100 is slowly added with stirring under nitrogen, the temperature being kept below 120° C. throughout the addition. Following the addition, the temperature of the contents of the flask is maintained at 120° C. for 30 minutes. Then the temperature is increased to 144° C. and maintained at 144° C. for an additional six hours. The reaction is considered to be complete when the acid number is below 10.

### EXAMPLE 4

### Preparation of Ethyleneamine E-100 Triamide Amphoteric Surfactant

To a 3-neck 1 L round bottom flask equipped with a mechanical stirrer, nitrogen purge, and addition funnel is charged 120.6 grams of propylene glycol and 98.3 grams (0.1 moles) of the oleic acid triamide of TEPA prepared from example 3 above. The contents of the flask are heated with stirring to 90° C. until the contents became homogeneous. 6.5 grams (0.090 mole) of acrylic acid are added slowly, and the contents of the flask are maintained at 105° C. for 3 hours. Alternatively, the reaction may be terminated when at least 90% of the acrylic acid has reacted, as determined by quantitative IR spectroscopy.

### Softness Tests for Tissue Paper

One important aspect of tissue paper for use in personal care such as facial tissue and bathroom tissue is the softness of such papers. In order to evaluate the effect of a compound according to the present invention, several test solutions were made up as follows:
Sample 1: 48% (TEPA+3 moles oleic acid+2 moles acrylic acid) 52% propylene glycol.
Sample 2: 48% (TEPA+2.5 moles oleic acid+1.5 moles acrylic acid) 52% propylene glycol.
Sample 3: 48% (TEPA+2 moles oleic acid+2 moles acrylic acid) 52% propylene glycol.
Sample 4: 48% (TEPA+2 moles oleic acid+1 moles acrylic acid) 52% propylene glycol.
Sample 5: 48% (TEPA+3 moles oleic acid+1 moles acrylic acid) 52% propylene glycol.
Sample 6: 70% of sample 1 mixed with 30% of SURFONIC® E-400 MO (“mono-oleate”).
Sample 7: 70% of sample 2 mixed with 30% of SURFONIC® E-400 MO.
Sample 8: 70% of sample 3 mixed with 30% of SURFONIC® E-400 MO.
Sample 9: 70% of sample 4 mixed with 30% of SURFONIC® E-400 MO.
Sample 10: 70% of sample 5 mixed with 30% of SURFONIC® E400 MO.
Sample 11: pure SUFURONIC® E-400 MO (SURFONIC products are available from Huntsman Corporation)
Control 1: 48% (diethylenetetramine “DETA”+2 moles TOFA (tall oil fatty acid)+1 mole acrylic acid)+52% propylene glycol.
Control 2: 70% of control 1+30% SUFRONIC® E-400 MO.

In the above samples, the terminology reminiscent of “(TEPA+2 moles oleic acid+2 moles acrylic acid)” means the amphoteric surfactant produced by reacting TEPA with 2 moles of oleic acid, and subsequently reacting the product thereof with 2 moles of acrylic acid. The various compositions descried above in samples 1-5 were prepared by simple mixing of the specified amount of glycol and amphoteric surfactant. Similarly, for examples 6-10 the specified amounts of materials were blended together. SUFRONIC® E-400 MO is an ethoxylated oleic acid surfactant available from Huntsman Company LLC of Houston, Tex.

Solutions for treating tissue paper were prepared by making up a 1.0% solution of each of the above samples in water. Evaluations of the effect of each solution were made by immersing a swatch of untreated tissue in each of the 1.0% aqueous solutions containing the material in the samples above. The treated tissue swatches were held in the solution for one minute, and withdrawn. The treated tissue swatches were then dried in an oven at 25° C. The tissues so treated were evaluated for their softness to the touch by several members of our research staff and each given a rating based on the scale: 0=poor/harsh texture; 1=fair; 2=good; 3=very good; 4=excellent/very soft texture. The results of the softness testing is tabulated in the table I below:

TABLE I
softness feel test results

| Sample ID | Softness |
|---|---|
| DI Water | 0 |
| Sample 6 | 2.4 |
| Sample 7 | 2.4 |
| Sample 8 | 1.2 |
| Sample 9 | 1.8 |
| Sample 10 | 3.8 |
| Sample 11 | 1.4 |
| Sample 5 | 4.0 |
| Control 1 | 2.5 |
| Control 2 | 2.4 |

Sample 6 and sample 7 are comparable to the prior art; however, sample 10 and sample 5 are superior to the prior art. In the graph below is the surface response curve for the above samples. It can be seen from the contour plot in FIG. 1 of the softness test results that the maximum performance occurs with 3 moles of oleic acid and 1 mole of acrylic acid.

Consideration must be given to the fact that although this invention has been described and disclosed in relation to certain preferred embodiments, obvious equivalent modifications and alterations thereof will become apparent to one of ordinary skill in this art upon reading and understanding this specification and the claims appended hereto. Accordingly, the presently disclosed invention is intended to cover all such modifications and alterations, and is limited only by the scope of the claims which follow.

1) A composition of matter useful for treating paper, textiles, and human skin comprising an amphoteric surfactant represented by the formula: 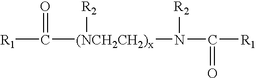
in which x is any integer selected from the group consisting of: 4, 5, and 6;
R₁ in each occurrence is independently any alkyl group having between 5 and 25 carbon atoms, whether straight-chain, branched, cyclic, saturated or unsaturated;
R₂ in each occurrence is independently selected from the group consisting of: 1) hydrogen; 2) any saturated or unsaturated aliphatic mono- or di-carboxylic acid moiety having one or more carboxyl functional groups and having one or more straight-chain or branched, saturated or un-saturated aliphatic chains containing from 2 to 20 carbon atoms; 3) a saturated or unsaturated aliphatic mono sulfonic acid moiety comprising an —SO₃H functional group and having one or more straight-chain or branched, saturated or un-saturated aliphatic chains containing from 2 to 20 carbon atoms; and 4) a radical of the formula: 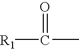
in which R₁ is defined as above.

2) A composition according to claim 1 wherein R₁ in each occurrence may be independently derived from a carboxylic having an unsaturated alkyl carbon bond.

3) A composition according to claim 1 wherein R₂ in each occurrence is independently derived from an acid selected from the group consisting of: acrylic acid, maleic anhydride, vinyisulfonic acid, allylsulfonic acid, 2-methyl vinyl sulfonic acid, and maleic acid.

4) A composition of matter useful for treating paper, textiles, and human skin comprising a mixture of at least two components each of which comprise different amphoteric surfactants which are represented by the formula: 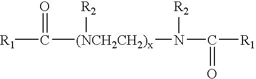
in which x is any integer selected from the group consisting of: 4, 5, and 6;
R₁ in each occurrence is independently any alkyl group having between 5 and 25 carbon atoms, whether straight-chain, branched, cyclic, saturated or unsaturated; R₂ in each occurrence is independently selected from the group consisting of: 1) hydrogen; 2) any saturated or unsaturated aliphatic mono- or di-carboxylic acid moiety having one or more carboxyl functional groups and having one or more straight-chain or branched, saturated or un-saturated aliphatic chains containing from 2 to 20 carbon atoms; 3) a saturated or unsaturated aliphatic mono sulfonic acid moiety comprising an —SO₃H functional group and having one or more straight-chain or branched, saturated or un-saturated aliphatic chains containing from 2 to 20 carbon atoms; and 4) a radical of the formula: 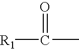
in which R₁ is defined as above.

5) A composition according to claim 4 wherein one of the components of said mixture has the structure: 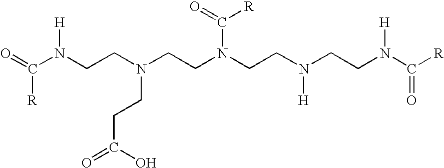
in which R in each occurrence R is independently any alkyl group having between 5 and 25 carbon atoms, whether straight-chain, branched, cyclic, saturated or unsaturated.

6) A composition according to claim 5 in which the carboxylic acid appendage on the nitrogen atom is derived from acrylic acid.

7) A composition according to claim 4 wherein one of the components of said mixture has the structure: 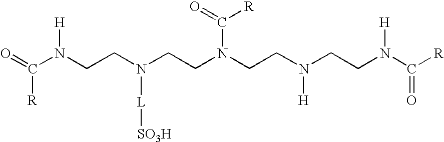
in which R in each occurrence is independently any alkyl group having between 5 and 25 carbon atoms, whether straight-chain, branched, cyclic, saturated or unsaturated, and in which L is any alkyl group having any number of carbon atoms between about 2 and about 20, whether straight-chain, branched, or cyclic.

8) A composition according to claim 4 wherein said mixture comprises:
a) a first amphoteric surfactant, having a value for x of 4;
b) a second amphoteric surfactant, having a value for x of 5;
c) a third amphoteric surfactant, having a value for x of 6,
said first amphoteric surfactant being present in any amount between 8.0% and 20.0%; said second amphoteric surfactant being present in any amount between 25.0% and 45.0%; and said third amphoteric surfactant being present in any amount between 35.0% and 60.0%, wherein said percentages are calculated on a weight basis with respect to all of the amphoteric surfactants present which are defined by said formula.

9) A process for treating a substrate comprising the steps of:
a) providing an aqueous composition that comprises an amphoteric surfactant according to claim 1; and
b) contacting said substrate with said aqueous composition.

10) A process according to claim 9 wherein the concentration of said amphoteric surfactant is any concentration between 0.10% and 5.00% by weight based upon the total weight of said aqueous composition.

11) A process for treating a substrate comprising the steps of:
a) providing an aqueous composition that comprises an amphoteric surfactant according to claim 4; and
b) contacting said substrate with said aqueous composition.

12) A process according to claim 11 wherein the concentration of said amphoteric surfactant is any concentration between 0.10% and 5.00% by weight based upon the total weight of said aqueous composition.

13) A process for treating a substrate comprising the steps of:
a) providing an aqueous composition that comprises an amphoteric surfactant according to claim 7; and
b) contacting said substrate with said aqueous composition.

14) A process according to claim 13 wherein the concentration of said amphoteric surfactant is any concentration between 0.10% and 5.00% by weight based upon the total weight of said aqueous composition.

15) A composition according to claim 1 further comprising at least one fabric.

16) A composition according to claim 1 further comprising at least one paper.

17) A composition according to claim 1 further comprising at least one tissue paper for personal care.

18) A composition according to claim 1 further comprising a hair care product.

19) A composition according to claim 1 further comprising a skin care product.

20) A composition according to claim 1 further comprising at least one textile.
